(19) United States
(12) Patent Application Publication
(10) Pub. No.: US 2002/0115738 A1
(43) Pub. Date: Aug. 22, 2002

(54) Functional breakable crosslinkers, and polymer networks thereof

(76) Inventors: Tohei Moritani, Coram, NY (US); Carmen Alvarez-Lorenzo, Santiago de Compostela (ES)

Correspondence Address: FOLEY HOAG LLP, PATENT GROUP, 155 SEAPORT BOULEVARD, BOSTON, MA, 02110, US

(21) Appl. No.: 10/068,550

(22) Filed: Feb. 6, 2002

Related U.S. Application Data
(60) Provisional application No. 60/267,900, filed on Feb. 9, 2001.
(60) Provisional application No. 60/299,981, filed on Jun. 21, 2001.

(51) Int. Cl. G01N 33/543; C08F 2/00

(52) U.S. Cl. 521/142

(57) ABSTRACT

This invention relates to functional breakable crosslinkers and polymer networks made of functional breakable crosslinkers. The polymer networks show high selectivity or molecular recognition and are suitable for the use in separation materials and sensors. A representative functional crosslinker is 2,3-Dihydroxy-N,N,N′N′-tetramethyl-N,N′-bis{3-[(2-methylacryloyl)amino]propyl}-1,4-butanediaminium dihalide (“Imprinter-Q”). Imprinter-Q comprises three functional parts: two polymerizable double bonds; two cationic groups; and a 1,2-glycol link between the cationic groups that is easily cleavable. Imprinter-Q can be polymerized with other polymerizable monomers and cross-linkers to obtain polymer networks. After breaking the 1,2-glycol bond, the polymer network has receptor sites that present high affinity for divalent anionic molecules or ions.

### RELATED APPLICATIONS

This application claims the benefit of priority under 35 U.S.C. section 119(e) to U.S. Provisional Patent Application Serial No. 60/267,900, filed Feb. 9, 2001; and U.S. Provisional Patent Application Serial No. 60/299,981, filed Jun. 21, 2001; these applications are hereby incorporated by reference in their entirety.

### GOVERNMENT SUPPORT

The invention was made with support provided by the National Science Foundation (DMR-9616791); therefore, the government has certain rights in the invention.

### BACKGROUND OF THE INVENTION

There have been a number of attempts to design synthetic polymers that show the highly specific molecular recognition of antibodies while keeping the folding/unfolding behavior of proteins. Vlatakis, G.; Andresson, L. I.; Müiller, R.; Mosbach, K. Nature 1993, 361, 645-647; Tanaka, T.; Wang, C.; Pande, V.; Grosberg, A. Yu.; English, A.; Masamune, S.; Gold, H.; Levy, R.; King, K. Faraday Discuss. 1996, 102, 201-206; Pande, V. S.; Grosberg, A.Yu.; Tanaka, T. Physica D 1997, 107, 316-321; Andersson, H. S.; Nicholls, I. A. In: Molecularly Imprinted Polymers. B. Sellergren (Ed.). Elsevier, Amsterdam 2001, pp. 1-19. For this purpose, Wulff, Mosbach, and others developed an imprinting technology where receptor sites are created by polymerization in the presence of target molecules. The materials formed by applying this technology present high affinity and selectivity to the desirable target molecules. Wulff, G.; Sarhar, A.; Zabrocki, K. Tetrahed. Lett. 1973, 44, 4329-4332; Wulff, G.; Vietmeier, J.; Poll, H.G. Makromol. Chem. 1987, 188, 731-740; Skudar, K.; Brüggemann, O.; Wittelsberger, A.; Ramström, O. Anal. Commun. 1999, 36, 327-311; Yilmaz, E.; Mosbach, K.; Haupt, K. Anal. Commun. 1999, 36, 167-170. However, since the backbone structure of the plastic matrix cannot be modified, the affinity cannot be not altered by changes in conformation. In contrast, proteins are basically flexible and able to change their affinity for the target depending on external stimuli. Molecular recognition is believed to occur through a specific folding of the protein due to van der Waals interactions, hydrogen bonds, electrostatic forces or hydrophobic interactions. As a model of this protein folding, polymeric gels that experiment a reversible phase transition depending on the environmental conditions are particularly useful as a basis to create flexible and re-naturable gels. Pande, V.S.; Grosberg, A. Yu.; Tanaka, T. Physica D 1997, 107, 316-321; Watanabe, M.; Akahoshi, T.; Tabata, Y.; Nakayama, D. J Am. Chem. Soc. 1998,120, 5577-5578.

To recognize specific target, these gels also have to show a “conformational imprinting effect” as a consequence of the following three steps (FIG. 1): a) Polymerization carried out after the monomers are allowed to equilibrate their spatial arrangement. The resulting polymer should be in the lowest energy conformation. This will be the driving force to memorize the conformation of the network and the location of functional groups, which form the receptor centers; b) Breaking the interaction between the functional groups that form each receptor site destroying the initial conformation. c) Adding specific molecules that can interact with the functional groups, so that the memory of the conformation is recalled by reconstructing the original conformation of the gel.

Based on these ideas, weakly cross-linked gels synthesized in the presence of target molecules have been prepared imitating the usual imprinting technology. Although losing in selectivity with respect to the imprinted plastics, it was possible to gain in control of affinity through changes in the conformation induced by external stimuli. Recently, Alvarez-Lorenzo et al. have shown for the first time a conformational imprinting effect in N-isopropylacrylamide (NIPA)-lead dimethacrylate gels that can reversibly swell and shrink in response to temperature. Alvarez-Lorenzo, C.; Guney, O.; Oya, T.; Sakai, Y.; Kobayashi, M.; Enoki, T.; Takeoka, Y.; Ishibashi, T.; Kuroda, K.; Tanaka, K.; Wang, G.; Grosberg, A.Yu.; Masamune, S.; Tanaka, T. “Polymer Gels That Memorize Elements of Molecular Conformation”, Macromolecules 2000, 33, 8693-8697. After washing lead out and swelling, the affinity for divalent ions disappeared. When the gels were shrunken again, the affinity was recovered, showing a complete recall of the original conformation. Control gels made using randomly distributed methacrylic acid monomers showed a much lower affinity. The success of the imprinting can be attributed to the fact that the degree of dissociation between lead and two methacrylate molecules during polymerization is negligible, and therefore lead plays the role of strongly fixing pairs of methacrylates. Alvarez-Lorenzo, C.; Guney, O.; Oya, T.; Sakai, Y.; Kobayashi, M.; Enoki, T.; Takeoka, Y.; Ishibashi, T.; Kuroda, K.; Tanaka, K.; Wang, G.; Grosberg, A. Yu.; Masamune, S.; Tanaka, T. J Chem. Phys. 2001, 114, 2812-2816.

The development of artificial systems able to mimic the molecular recognition carried out by proteins and enzymes is receiving increased attention; for such a systems would have an enormous potential of applications (Bayerl et al., U.S. Pat. No. 6,051,372; Johnson et al., U.S. Pat. No. 6,140,471). One of the most common approaches to the synthesis of host molecules, which can recognize target guest species, is a polymerization technique in which the target molecules are used as template, known as molecular imprinting. Molecular imprinting is a process for preparing materials that are selective for a particular compound (the template molecule) or set of related compounds. To control the sequence and spatial arrangement of the monomers, the template molecule and the functional monomers are allowed to associate before polymerisation through reversible covalent bonds (Wulff, G. Angew. Chem. Int. Ed. Engl. 1995, 34:1812) or through non-covalent or metal coordination interactions (Vlatakis et al. Nature 1993, 361:645). The utility of these materials for analytical determinations or to design sensors (Sasaki et al., U.S. Pat. No. 6,057,377) has been broadly proved.

In all cases, the networks prepared using molecular imprinting incorporate a high proportion of functional monomers (to adsorb efficiently an important amount of target molecules), and an even higher proportion of cross-linker (to keep the spatial arrangement of the functional groups after the target is removed). The success of the imprinting depends on the stability of the complexes and solubility of the complexes template/functional monomers formed before polymerization. If the molar ratio in the complex is not the appropriate or if the complex dissociates to some extent during polymerisation, the functional monomers would be far apart from both the template and each other, resulting in a small difference between imprinted and non-imprinted materials. This effect has been observed in imprinted gels prepared with divalent salts of methacrylic monomers in different solvents. Alvarez-Lorenzo, C.; Guney, O.; Oya, T.; Sakai, Y.; Kobayashi, M.; Enoki, T.; Takeoka, Y.; Ishibashi, T.; Kuroda, K.; Tanaka, K.; Wang, G.; Grosberg, A. Yu.; Masamune, S.; Tanaka, T. J Chem. Phys. 2001, 114, 2812-2816. Successful results were obtained using lead methacrylate since this monomer presents a very low degree of dissociation and can be dissolved in non-polar medium.

The strength of the interaction between lead and the methacrylate monomers is the key of the imprinting effect in weakly cross-linked gels. It has been demonstrated that gels prepared with this compound and a monomer that allows the gel to swell and shrink in response to environmental stimuli, can develop their affinity for divalent metals under specific circumstances. Oya, T. et al. Science 1999, 286, 1543-1545; Alvarez-Lorenzo et al. Macromolecules 2001, 33, 8693. In the swollen state, the methacrylate units are far apart, the recognition sites are broken, and the gel presents a low affinity for a divalent metal. In the shrunken state, the methacrylate units can reform the receptor centers, and the affinity for divalent metals increases dramatically. In this case, the temperature can switch on and off the adsorption process. A similar gel prepared with randomly distributed methacrylic monomers (i.e. non-imprinted) presents a much lower affinity for divalent metals in both swollen and collapsed states. Alvarez-Lorenzo et al. Macromolecules 2001, 33, 8693.

Since it is difficult to find salts of monomers that can be dissolved without dissociation, the development of new functional monomers that can act as a cross-linker during polymerization, in which the ionic groups were strongly connected by a breakable bond is taught by the present invention. The linkage can be broken after polymerization to obtain pairs of ionic groups with the same charge. Since the members of each pair are close together, they can capture target molecules through multiple-point ionic interactions.

### SUMMARY OF THE INVENTION

The present invention provides a new type of monomer called Imprinters. Co-polymerization of an Imprinter with a stimuli-sensitive monomer forms a gel with receptor sites of reversible affinity. One such Imprinter, Imprinter-Q, is a dimeric monomer that has two cationic groups linked by a 1,2-glycol bond, which is easily cleavable.

In another embodiment, the present invention is a co-polymerization of an Imprinter and a stimuli-sensitive monomer in the presence of a cross-linker. Weakly cross-linked gels have been prepared using Imprinter-Q, N-isopropylacrylamide, and cross-linker N,N′-methylenebisacrylamide. After breaking the 1,2-glycol link, the members of each cationic pair are close together and can capture target molecules via a multiple-point electrostatic interaction. The higher affinity of these gels for disodium nitroisophthalate (NPA) in comparison with control gels, which were prepared with randomly distributed cationic groups, proved that the gels prepared with Imprinter-Q memorized the position of the pairs of cationic groups after swelling and re-shrinking. The control gels showed frustration in forming pairs and their affinity for NPA decayed exponentially as a function of cross-linker concentration. In contrast, such frustration was completely removed for the imprinted gels, showing that memorization had been achieved. This “conformational imprinting effect” was tested for several concentrations of Imprinter-Q and permanent cross-linkers.

This invention further includes novel functional breakable crosslinkers and polymer networks made of functional breakable crosslinkers. The polymer networks show high selectivity or molecular recognition and are suitable for the applications of separation materials and sensors. A typical novel functional crosslinker is 2,3-Dihydroxy-N,N,N′N′-tetramethyl-N,N′-bis{3-[(2-methylacryloyl)amino]propyl}-1,4-butanediaminium dihalide (Imprinter-Q). Imprinter-Q has three functional parts: two polymerizable double bonds, two cationic groups, and a 1,2-glycol link between the cationic groups that is easily cleavable. Imprinter-Q can be polymerized with other polymerizable monomers and cross-linkers to obtain polymer networks. Mar After breaking the 1,2-glycol bond, the network has receptor sites that present high affinity for divalent anionic molecules or ions. The network shows excellent molecular recognition to divalent anionic molecules.

Imprinter-Q has been found to give weakly cross-linked gels that show higher affinity for divalent anionic molecules than that of gels made with randomly distributed cationic groups. This observation can be attributed to the imprinting or the memorizing of the conformation during polymerization. This behavior is similar to gels made using lead methacrylate previously reported by Alvarez-Lorenzo et al. (2000). From these two cases, one can conclude that dimeric monomers with two functional groups strongly held together by cleavable links such as a lead ion or a 1,2-glycol bond are effective for making imprinted gels. The concept of Imprinters is expected to open the technology for producing more sophisticated gels with multiple imprinted positions, which can exhibit molecular recognition by mimicking the folding and unfolding of proteins.

### BRIEF DESCRIPTION OF THE FIGURES

FIG. 1 shows a schematic representation of the conformational imprinting effect.

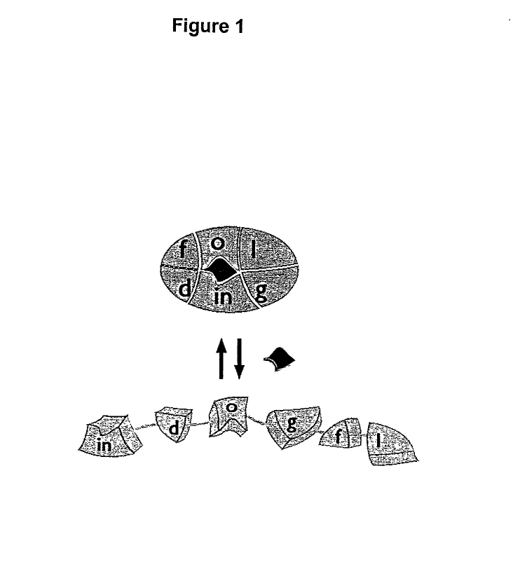

FIG. 2 provides a method for the synthesis of Imprinter-Q and preparation of the Imprinter-Q/NIPA gels.

FIG. 3 shows a preparation of gels made with randomly distributed cationic groups.

FIG. 4 represents a ¹H NMR spectra of Imprinter-Q in deuterium oxide before and after treatment to break the 1,2-glycol bond.

FIG. 5 depicts a ¹³C NMR spectra of Imprinter-Q in deuterium oxide before and after treatment to break the 1,2-glycol bond.

FIG. 6 illustrates a dependence of the saturation value, S, the affinity per adsorption site, K, and an overall affinity, SK, on the concentration of cationic groups, for Imprinter-Q gels and MAPTAC gels.

FIG. 7 shows the influence of temperature in the release and readsorption process of NPA by imprinted gels prepared with different concentrations of Imprinter-Q.

FIG. 8 depicts the dependence of the overall affinity, SK, of the Imprinter-Q and MAPTAC gels on the cross-linking density (BIS), in the swollen (A) and shrunken (B) states.

FIG. 9 provides a ¹H NMR spectra of Imprinter-Q in deuterium oxide.

FIG. 10 is a ¹³C NMR spectra of Imprinter-Q in deuterium oxide.

FIG. 11. shows reversible adsorption of disodium 5-nitrophthalate by Imprinter-Q/NIPA gel and the switching effect of the temperature.

FIG. 12 depicts the influence of the cross-linking degree on the affinity of Imprinter-Q/NIPA gels compared to NIPA gels prepared with randomly distributed cationic groups.

### DETAILED DESCRIPTION OF THE INVENTION

This invention relates to a new polymerizable material useful for preparing synthetic polymer networks and hydrogels that show molecular recognition. In particular embodiments, this invention provides imprinted cationic pairs in hydrogels.

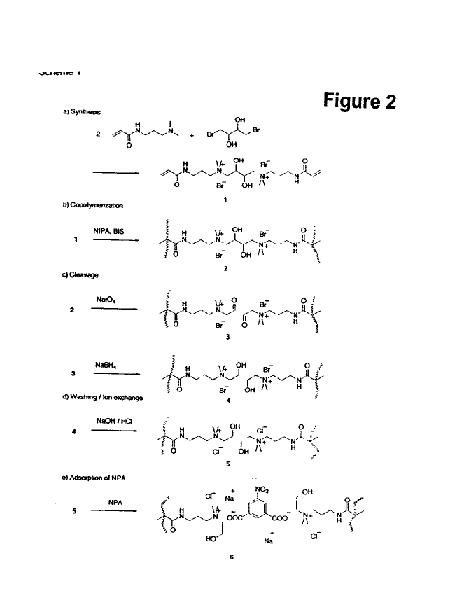

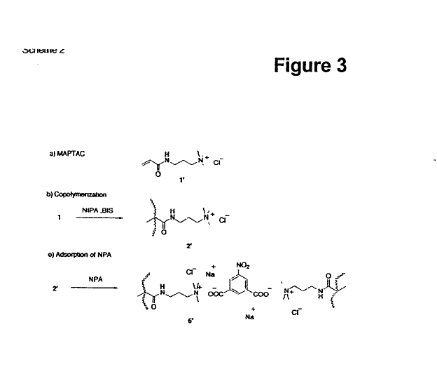

In order to extend this behavior to a more generalized technology, a new series of functional monomers have been designed, called “Imprinters”. An Imprinter is a molecule that has three functional parts: two or more polymerizable double bonds, two or more functional groups, and a link connecting the functional groups that is easily cleaved afterwards, such as a 1,2-glycol structure. Imprinter-Q (2,3-Dihydroxy-N,N,N′N′-tetramethyl-N,N′-bis {3-[(2-methylacryloyl)aminopropyl)-1,4-butanediaminium dibromide) has two quarternary ammonium groups as cationic groups. Imprinter-Q is a novel substance that has been unknown in the literature (FIG. 2). By co-polymerizing Imprinter-Q and a stimuli-sensitive monomer such as N-isopropylacrylamide (NIPA), it is possible to develop hydrogels that are able to adsorb charged molecules and simultaneously have switching capacities. The linkage between the quaternary ammonium groups of each Imprinter-Q is broken after polymerization to obtain pairs of cationic groups. Since the members of each pair are close together, they can capture target molecules through multiple-point ionic interactions. The NIPA component of the gel is responsible for the swelling at temperatures lower than 33° C. and collapsing when the temperature is raised. Schild, H. G. Prog. Polym. Sci. 1992,17,163-249. This gel was compared with a reference gel made of randomly distributed cationic groups (FIG. 3).

A. Definitions

For convenience, the definitions of several terms used repeatedly throughout this document are collected here.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

The abbreviation “BIS” as used herein stands for N,N′-methylenebis (acrylamide) and is used herein as a polymer cross-linker

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means -SH; the term “hydroxyl” means —OH; and the term “sulfonyl” means —SO₂—.

The terms “amine” and “amino” are art-recognized and include both unsubstituted and substituted amines, e.g., a moiety that may be represented by the general formulas:

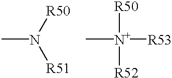

Wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

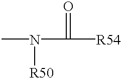

Wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

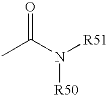

Wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

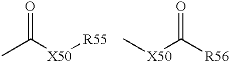

Wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

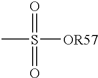

In which R57 is an electron pair, hydrogen, alkyl, cycloalkyl, or aryl.

The term “sulfate” is art recognized and includes a moiety that may be represented by the general formula:

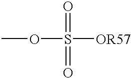

In which R57 is as defined above.

The term “sulfonamido” is art recognized and includes a moiety that may be represented by the general formula:

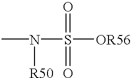

In which R50 and R56 are as defined above.

The term “sulfamoyl” is art-recognized and includes a moiety that may be represented by the general formula:

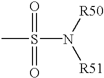

In which R50 and R51 are as defined above.

The term “sulfonyl” is art recognized and includes a moiety that may be represented by the general formula:

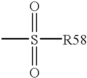

In which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

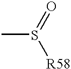

In which R58 is defined above.

The term “phosphoramidite” is art recognized and includes moieties represented by the general formulas:

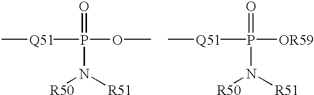

Wherein Q51, R50, R51 and R59 are as defined above.

The term “phosphonamidite” is art recognized and includes moieties represented by the general formulas:

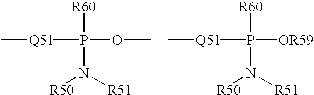

Wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

B. Imprinters

Imprinters of the present invention are compounds that have three characteristics: two or more polymerizable double bonds, two or more functional groups, and a link connecting the two functional groups that is easily cleaved. Examples of Imprinters are presented below according to the type of cleavable link and functional groups they possess.

1,2-Glycol Link

Quaternary Ammonium Groups

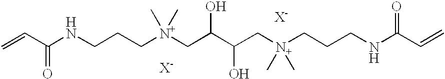

Imprinter Q-2,3-Dihydroxy-N,N,N′N′-tetramethyl-N,N′-bis{3-[(2-methylacryloyl) amino]propyl}-1,4-butanediaminium dihalide

This Imprinter has been polymerized with (meth)acrylic monomers and divinyl monomers to make hydrogels. The 1,2-glycol was cleaved under the mild conditions with the treatment of sodium periodate and the resulting aldehyde groups were reduced with sodium borohydride to give the more stable alcohol groups within the gels:

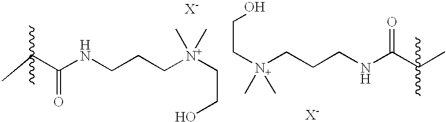

The resulting gels have been found to show higher affinity to substances having negative charge like sodium 5-nitroisophtalate (NPA). See FIG. 2.

Secondary Amino Groups

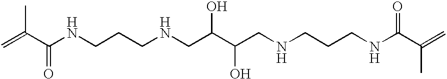

Imprinter-A

Carboxylic Acid Groups (hidden)

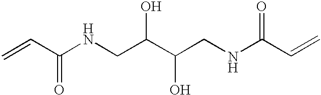

This Imprinter has been polymerized together with (meth)acrylic monomers and divinyl monomers to make polymer networks or hydrogels. The 1,2-glycol links is cleaved under the mild conditions with the treatment of sodium periodate. The resulting aldehyde group can be converted to a carboxyl group with the treatment of oxdizing agent like hydrogen peroxide.

The imprinted pair of carboxyl groups has been created inside gels as shown below.

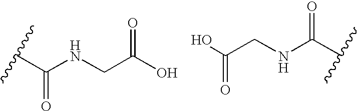

Hydrophobic Alkyl Groups

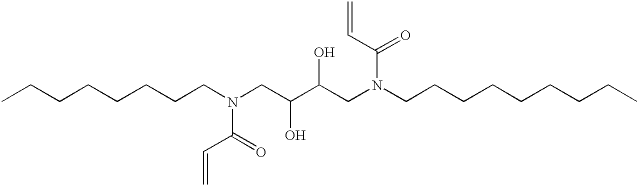

Imprinter-H

Aromatic Groups Such as Pyranyl

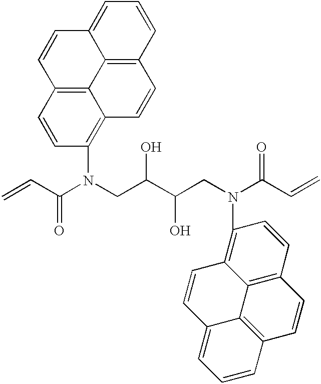

Imprinter-Py

Heterocyclic Groups Such as Imidazole and.

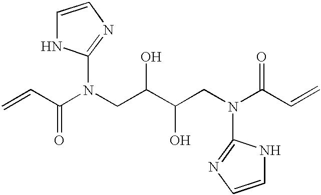

Imprinter-Im

Methylimidazole

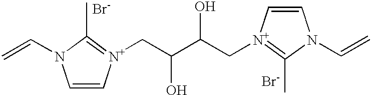

Imprinter-QMVI

Disulfide Link

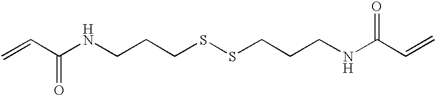

This Imprinter has been polymerized together with (meth)acrylic monomers and divinyl monomers to make polymer networks or hydrogels. The disulfide link is cleaved under the mild conditions with the treatment of sodium borohydrate. The resulting thiol group can be converted

To sulfonium groups with the treatment of an oxdizing agent like hydrogen peroxide. The imprinted pair of sulfonium groups have been created inside gels as shown below.

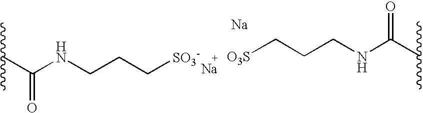

C. Other Components Used to Form the Hydrogels

Polymerization is carried out using a mixture of one or more functional crosslinker monomers (Imprinters) shown above, structural monomers, other functional monomers, crosslinker(s), polymerization initiators and solvent.

Structural Monomers

Structural monomers include, but are not limited to, any one of the following, or mixtures thereof: methyl (meth)acrylate, ethyl (meth)acrylate, propyl (meth)acrylate, butyl (meth)acrylate, other alkyl (meth)acrylate, (meth)acrylamide, N-methyl(meth)acrylamide, N-ethyl(meth)acrylamide, N-n-propyl(meth)acrylamide, N-isopropyl(meth)acrylamide, N-isobutyl(meth)acrylamide, other N-alkyl (meth)acrylamide, methyl (meth)acrylate, ethyl (meth)acrylate, n-propyl (meth)acrylate, isopropyl (meth)acrylate, other alkyl (meth)acrylate, ethylene glycol monomethacrylate, styrene, vinyl acetate, or vinyl alcohol.

Other Functional Monomers

Other functional monomers include, but are not limited to, any one of the following, or mixtures thereof: carboxyl monomers like (meth)acrylic acid, crotonic acid, maleic acid, fumaric acid, itoconic acid, or vinylbenzoic acid. Sulfonic monomers like 2-acrylamido-2-methyl-1-propane sulfonic acid, N-(3 aminopropyl) methacrylamide, methacrylamidopropyltrimethylammonium chloride, vinyl pyridine, vinyl imidazole, or N-vinyl-2-methylimidazole.

Crosslinkers

Crosslinkers include, but are not limited to, any one of the following, or mixtures thereof: ethylenglycol di(meth)acrylate, N,N′-methylene-bis-(meth)acrylamide, N,N′-phenylene-bis-(meth)acrylamide, trimethylpropane tri(meth)acrylate, pentaerythritol tri(meth)acrylate, trimethylpropane tri(meth)acrylate, or 1,4-divinylbenzene.

Polymerization Initiators

Polymerization initiators include, but are not limited to, azobis (isobutyronitrile) or benzoyl peroxide.

Solvents

Solvents that can be used in the present invention include, but are not limited to water, methanol, ethanol, benzene, toluene, chloroform, dichloromethane, acetonitrile, acetone, tetrahydrofarane, dioxane, pyridine, dimethylsulfoxide, or dimethylformamide or mixtures thereof.

D. Preparation of Polymer Networks and Hydrogels

Polymerization.

Into the mixture of one or more functional crosslinker monomers shown above (Imprinter), structural monomers, other functional monomers, crosslinker(s), and solvent, is added a polymerization initiator. Preferably air is removed by introducing nitrogen gas and the temperature is increased.

Breaking the Breakable Bond

In case of the functional crosslinkers containing a 1,2-glycol structure, the materials after polymerization are treated with an oxidizing agent such as sodium periodate. Resulting materials are preferably treated with a reducing agent such as sodium borohydride in order to convert aldehyde group to more stable hydroxyl group. Resulting materials are preferably washed and purified with water. In case of the compounds where the breakable bond is disulfide link, the materials after polymerization are treated with a reducing agent such as sodium borohydride. Resulting materials are treated with a oxidizing agent such as hydrogen peroxide in order to convert thiol group to sulfonium group.

E. Advantages and improvements over existing methods devices, or materials

The present invention is the first example known to make imprinted pairs in polymer networks and hydrogels without using a template. Resulting polymer networks and hydrogels show high selectivity and molecular recognition.

F. Commercial Applications

The present invention can be applied in a number of applications such as solid phase extractions, chromatography columns, capillary electrochromatography, sensors, and controlled release.

G. Imprinter-Q Characterization

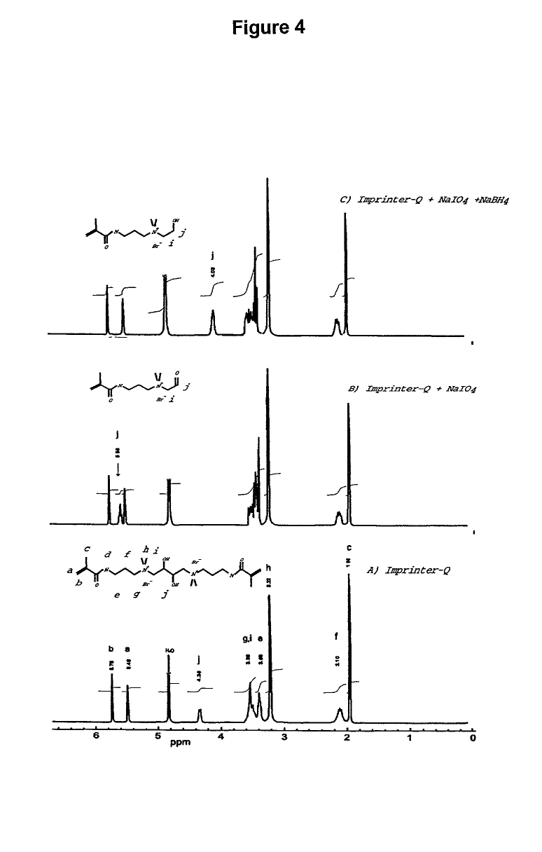

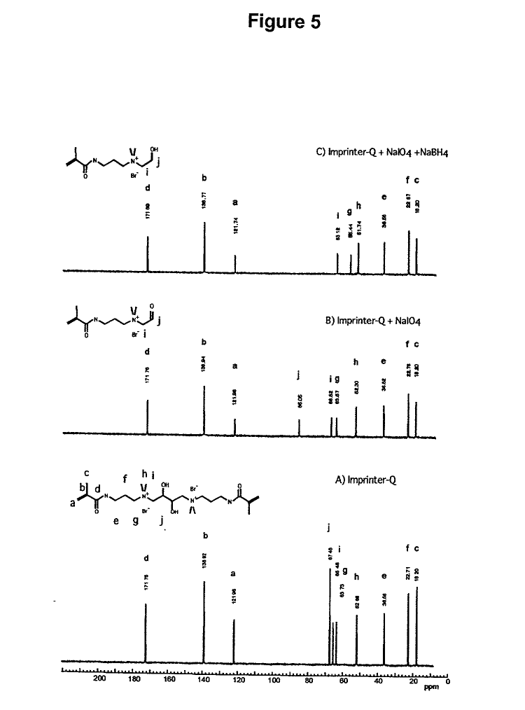

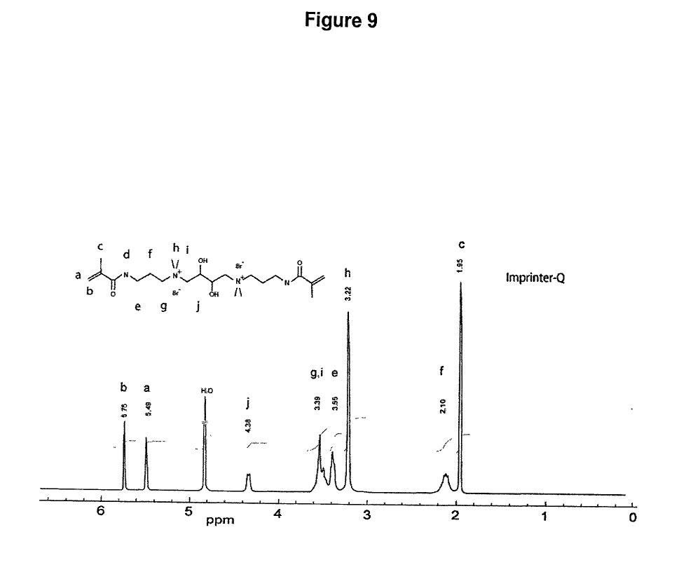

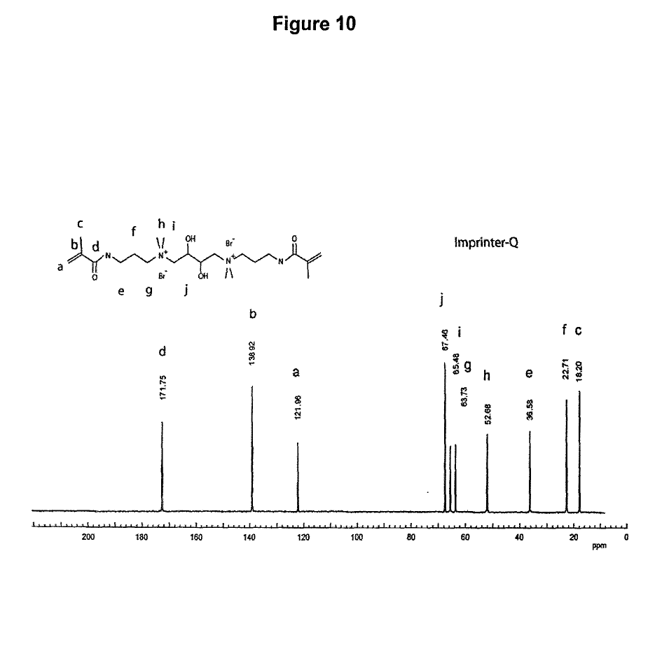

Imprinter-Q was synthesized by the reaction of N-(3-dimethylaminopropyl) methacrylamide and 1,4-dibromo-2,3-butanediol and purified (FIG. 2). ¹H NMR (FIG. 4-A and FIG. 9) and ¹³C NMR (FIG. 5-A and FIG. 10) spectra of Imprinter-Q in deuterium oxide as a solvent corroborate the chemical structure of Imprinter-Q and the absence of impurities. To cleave the 1 ,2-glycol bond in Imprinter-Q, the procedure commonly used to break this bond in the final conditioning of poly(vinylalcohol) was selected. Harris, H. E., Pritchard, J. G. J. Polym. Sci., Part A 1964, 2, 3673. After adding NaIO₄, aldehyde groups are obtained (spectra FIGS. 4-B and 5-B). Finally, in the presence of NaBH₄, the aldehyde groups are converted to stable alcohol groups (spectra FIGS. 4-C and 5-C). The main changes were observed for the lines assigned to methylene (i) and methine 0) protons, and carbons involved in 1,2-glycol linkage between two quaternary ammonium groups. The line j in the both figures can be assigned to the aldehyde proton and carbon although the observed chemical shifts, 5.6 ppm and 85,05 ppm in ¹H and ¹³C NMR, respectively, are abnormal: the usual values for an aldehyde are about 10 ppm and 200 ppm, respectively. These anomalous chemical shifts can be explained by the effects of other coexistent ionic species. In FIG. 4-C, the line at 4.03 ppm has an intensity double than that of the ones observed for the corresponding lines of j in FIGS. 4-A and 4-B. This line can be assigned to the methylene j proton generated by the reduction of aldehyde. The line of j is missing in FIG. 5-C. This can be attributed to the effects of iodine and boron compounds. From these results, it can be concluded that the reactions of cleavage of 1 ,2-glycol and reduction of aldehyde proceed quickly, almost within ten minutes.

H. Gel Behavior and Adsorption/Release Studies

The incorporation of Imprinter-Q to the gel during polymerization and the adequacy of the procedure used to break the 1 ,2-glycol bonds in the gels were initially tested as follows. First, a gel prepared only with NIPA and Imprinter-Q, without cross-linker BIS, swells in deionized water without dissolving, showing than Imprinter-Q is now acting as cross-linker itself Second, when this gel is transferred to 0.1IN NaIO₄ solution, it dissolved after 10 minutes, because of the breaking of the 1,2-glycol bonds. Third, using a cross-linked NIPA/MAPTAC gel (no Imprinter-Q) that 0.1N NaIO₄ does not damage other bonds of the polymeric skeleton was tested. No changes in the swelling degree of the gel were observed after 12 hours in 0.1N NaIO₄ solution. After these observations, all NIPA/Imprinter-Q gels used for the next experiments were treated for 30 minutes in 0.1N NaIO₄ to ensure complete breakage of the 1,2-glycol bonds.

Influence of Cationic Groups Content:

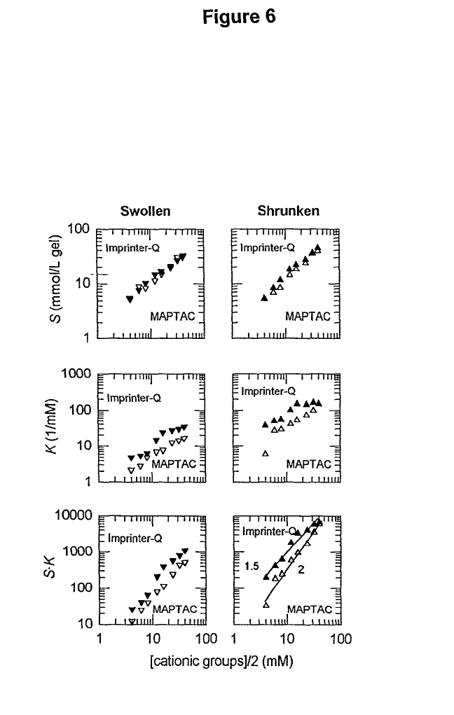

The adsorption of disodium 5-nitroisophthalate (NPA) by both Imprinter-Q and MAPTAC gels was well described by the Langmuir's isotherm as formulated in Eq. 2. The analysis of the dependence of the parameters S, K, and S K on the concentration of cationic groups in the gels (FIG. 6), for a fixed low concentration of cross-linker BIS, showed that:

a) Both Imprinter-Q and MAPTAC gels exhibit a linear relationship between the amount of ions needed to saturate the adsorption sites, S, and the concentration of the cationic groups in the polymer. In the swollen and in the shrunken state, the number of adsorption sites is approximately half the number of cationic groups that were incorporated during the synthetic procedure: S=[cationic groups]/2. This indicates that all cationic groups participate in forming adsorption sites for NPA molecules when the concentration of NPA is high enough.

b) The affinity per site, K, was larger for the Imprinter-Q gels than for the MAPTAC gels in both the swollen and shrunken states. This indicates that in the gels prepared with Imprinter-Q, the arrangement of the pairs of the cationic groups can create receptor sites of high affinity for a divalent anionic molecule like NPA. The affinity per site, K, was proportional to the concentration of cationic groups, and significantly lower in the swollen state than in the shrunken state. When the gel is swollen, the distance between the nearest cationic groups increase and the probability for pair formation decreases. The gel loses the imprinting conformation. In the shrunken state, the cationic groups can come close to each other again and the affinity for NPA increases almost I order of magnitude. This shows the destruction and reformation of NPA adsorption sites made of a pair of cationic groups.

c) The overall affinity of the gels to NPA ions, S K, was found to be higher in the Imprinter-Q gels than in the MAPTAC gels, except for the highest concentration of cationic groups. The difference in affinity between Imprinter-Q and MAPTAC gels is maximum when the concentration of adsorbing monomers (cationic groups) is lower than that of the cross-linker (40 mM). This can be understood by considering that if the concentration of adsorbing monomers is more than that of the cross-linker, there will be no frustration to from pairs, and thus no imprinting effect.

In the shrunken state, the slope of SK vs the concentration of cationic groups was lower for the Imprinter-Q gels than for the MAPTAC gets. This reflects the fact that the probability that randomly distributed monomers come into vicinity is proportional to the square of the adsorbing monomer concentration. In contrast, if the gel is synthesized non-randomly, the probability of a monomer to find a partner nearby becomes more higher than in the random case.

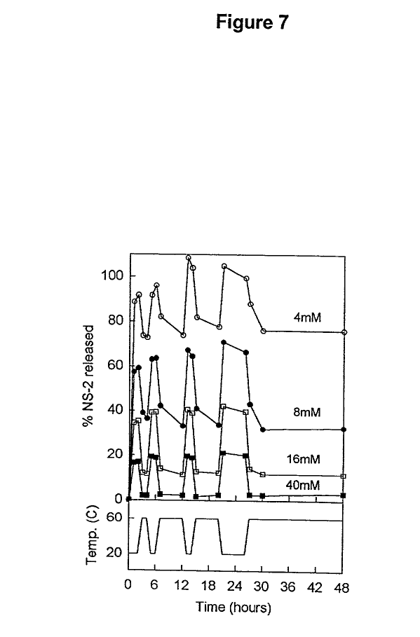

FIG. 7 shows the enormous effect of the temperature to switch on and off the adsorption ability of the Imprinter-Q gels. When the dry loaded gels are immersed in the aqueous medium, there is a fast hydration and swelling during which the gels release quickly NPA until the equilibrium is reached. Since the adsorption behavior of the gels follows the Langmuir model and the amount of NPA loaded in each gel depends on the proportion in cationic groups, the percentage of NPA released decreases as the proportion of cationic groups increases. However, the total amount of NPA released in the volume selected was higher for the gels more loaded, i.e. containing more cationic groups. It is greatly important to observe that when the temperature increases up to 60° C., the gels were able to re-adsorb a significantly high amount of the NPA previously released. This is a new behavior is not known to have been reported for NIPA gels. The temperature responsiveness of NIPA gels has been proposed by several authors as a way to create intelligent materials for controlled release (Hoffman et al., 1986; Bae and Kwon, 1998; Chung et al., 2000; Kim et al., 2000; Lee and Yen, 2000). In these previous systems, a substance entrapped in the NIPA network can diffuse out the gel in the swollen state, because of the increase in the porosity of the network. In contrast, an increase in temperature induces the network to collapse, which decreases dramatically its porosity, hindering the diffusion of the substance. Therefore, a pulsatile release behavior is obtained upon small changes in temperature. In the gels of the present invention, due to the affinity of the receptor groups for NPA, a change from the swollen to the shrunken state induces not only to stop the release but also to promote a readsorption process. This process occurs quickly and in a reproducible way after several temperature cycles. The MAPTAC gels presented a similar swelling/collapse behavior to the Imprinter-Q gels, however since the affinity for NPA is smaller than in the case of the Imprinter-Q gels, shrunken MAPTAC gels could not re-adsorb as much as the Imprinter-Q gels.

Influence of cross-linking degree:

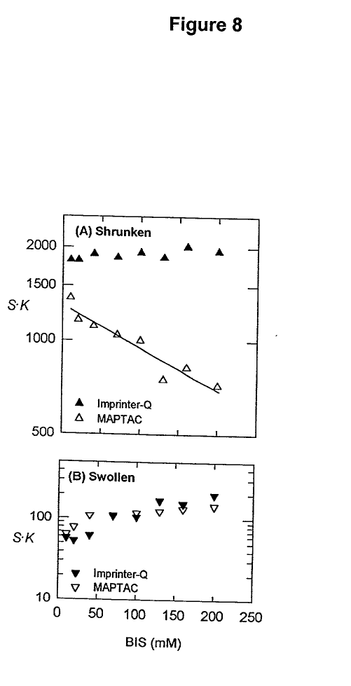

The effect of the cross-linking degree on the adsorption behavior of the MAPTAC and Imprinter-Q gels is shown in FIG. 8. In the shrunken state, the overall affinity, S-K, of the MAPTAC gels decayed exponentially as a function of cross-linker (BIS) concentration. The affinity of the Imprinter-Q gels was much larger than that of the MAPTAC gels and did not decrease with BIS. Similar phenomenon has been already observed in imprinted gels prepared using lead methacrylate and control gels made with methacrylic acid (Alvarez-Lorenzo et al., 2000). These observations represent a proof of frustration in the MAPTAC gels and its minimization in the Imprinter-Q gels. Alvarez-Lorenzo et al. Macromolecules 2000, 33, 8693; Enoki et al. Phys. Rev. Lett. 2000, 85, 5000. The exponential decay of the affinity of the MAPTAC gels can be understood as follows. The cationic groups in the gel can move rather freely within a certain volume determined by the cross-linking density. Indeed, it is established that below a certain length scale associated with the cross-link density, the gel behaves like a liquid, allowing the cationic groups to diffuse virtually freely. Grosberg, A. Yu.; Khokhlov, A. R. Statistical Physics of Macromolecules. AIP Press: New York, 1994; pp. 1-69. Beyond that length scale, the gel behaves as an elastic solid body, arid, in particular, the cationic groups cannot diff-use beyond a given distance. To make a simple estimate of this distance, 1, one may assume that each cationic group is at one end of a fictitious Gaussian chain with a length half that of the average polymer length between the nearest BIS cross-links (Alvarez-Lorenzo et al., 2000; Enoki et al., 2000),

l=nb=[NIPA]/[BIS]/2 (3)

Here n is the number of monomer segments of persistent length b. This fictitious Gaussian chain represents the restricted ability of the cationic groups to diffuse within a certain volume in the gel. It has been previously established that the affinity should be proportional to the probability for two adsorbing groups to meet, which is proportional to the Boltzmann factor of the entropy loss associated with the formation of one pair of adsorbing groups (Alvarez-Lorenzo et al., 2000; Enoki et al., 2000),

P=P₀exp(−R²/n·b²)=P₀exp(−c[BIS]/[MAA]^{⅔}) (4)

In contrast, for the imprinted gels there was no dependence of the affinity on BIS concentration (FIG. 8A). This is because the gel was synthesized using Imprinter-Q as a precursors of pairs of cationic groups. The Imprinter-Q gels have a much larger adsorption than that of MAPTAC gels because of the minimization of the frustration. If the Imprinter-Q gel did not memorize the position of the pairs of cationic groups after swelling and re-shrinking, a cationic group would have to find a new partner to form a pair, and the probability of forming such a pair would be the same as that in a randomly made gel. There would be no difference, then, between the NPA adsorption by the imprinted and the non-imprinted gels. One can therefore conclude that the excess NPA adsorption by the Imprinter-Q gel comes from the successfully memorized pairs.

Exemplification of the Invention

The invention now being generally described, it will be more readily understood by reference to the following examples, which are included merely for purposes of illustration of certain aspects and embodiments of the present invention, and are not intended to limit the invention.

### EXAMPLE 1

Synthesis of Imprinter-Q

Chemicals used in one method for synthesizing Imprinter-Q, include N-(3-dimethylaminopropyl) methacrylamide and 1,4-dibromo-2,3-butanediol from Sigma-Aldrich (WI), as well as methanol, isopropanol, ethyl acetate, dimethylsulfoxide, 2,2′-azobisisobutyronitrile (AIBN), and 5-nitroisophthalic acid (NPA). N-isopropylacrylamide (NIPA) and methacrylamidopropyl trimethylammonium chloride (MAPTAC) from Kohjin Co. Ltd. and Mitsubishi Rayon Co. Ltd. (Japan), respectively. N,N′-methylenebis(acrylamide) (BIS) from Blo-Rad Laboratories (CA).

To a stirred solution of N-(3-dimethylaminopropyl) methacrylamide (16.6 g) in methanol (120 mL), a solution of 1,4-dibromo-2,3-butanediol (11.4 g) in methanol (60 mL) was added. The reaction was carried out at 60° C. for 23 hours. Metanol was removed by evaporation. The remaining oily liquid was dissolved in isopropanol (120 mL). The solution was poured into a large amount of ethyl acetate (1800 mL). The precipitate was taken out by decantation and washed again with ethyl acetate. The solvent was evacuated in a rotary vacuum pump for 24 hours. White hygroscopic powder was obtained. Yield: 23.7 g (88%). ¹H and ¹³C NMR spectra were obtained using a Varian UNITY-300 spectrometer, operating at 300 MHz and 75 MHz, respectively. See FIGS. 9 and 10. After measuring the ¹H and ¹³C NMR spectra of the product in deuterium oxide (10 wt %, 0.7 mL), an aqueous solution of NaIO₄ (0.2N, 0.12 mL) was added in the NMR sample tube. Later, into the same sample tube, NaBH₄ (18 mg) was added and mixed for 10 minutes. The ¹ H and ¹³C NMR measurements after the addition of the chemicals were completed within ten minutes. See FIGS. 4a-c and 5a-c.

### EXAMPLE 2

Preparation of Gels

Gels were prepared by free radical polymerization using N-isopropylacrylamide (NIPA, 6M), Imprinter-Q and cross-linker BIS in dimethylsulfoxide. To study the influence of the proportion of functional groups, the cross-linker proportion (40 mM BIS) was fixed and used Imprinter-Q concentration ranged from 4 to 40 mM. For the cross-linker dependence study, the cross-linker BIS ranged from 10 mM to 200 mM, while Imprinter-Q concentration was fixed to 16 mM. After the addition of 2,2′-azobisisobutyronitrile (AIBN, 10 mM, initiator), the solutions were immediately transferred to test tubes in which glass capillaries (˜0.5 mm 1.d.) were placed. The solutions filled the capillaries, and were then degassed under vacuum for a few seconds. The polymerization was carried out at 60° C. for 24 hours. After gelation was completed, the gels were taken out the capillaries and consecutively washed with deionized water, 10 mM NaOH and 10 mM HCl, and deionized water for three days in each medium. To break the 1,2-glycol bond in the Imprinter-Q mers, the gels were introduced into 0.1N NaIO₄ for 30 minutes, rinsed with deionized water and transferred into 0.1N NaBH₄ for 30 minutes (Harris and Pritchard, 1964). Finally, the gels were washed with deionized water and 10 mM HCl, immersed in deionized water and collapsed at 60° C. to reduce the amount of water bonded. The gels were removed from the solution and dried under vacuum for one week.

Reference gels (i.e. non-imprinted) were synthesized as described above, using MAPTAC instead of Imprinter-Q.

### EXAMPLE 3

Characterization of the Gels

Swelling Degree. Equilibrium diameter d of the cylindrical gels in water were measured using a microscope equipped with a color video camera. The swelling degree was expressed as:

Swelling degree: V /V₀=(d /d₀)³ (1)

Where d₀ was the gel diameter upon polymerization.

Adsorption Studies. Pieces of cylindrical gel of dry weight 10 to 20 mg were placed in 4 mL disodium 5-nitroisophthalate (NPA) aqueous solutions (8 μM to 0.5 mM). The solutions also contained 1 mM NaCl to provide monovalent sodium ions to replace NPA molecules. The samples were kept swollen at 20′C or shrunken at 60° C. for 48 hours while being stirred. Equilibrium NPA concentration in the medium was measured spectrophotometrically at 266 nm. The amount of NPA adsorbed by the samples was then evaluated by the difference between the initial and the final concentrations.

The adsorption isotherms were analyzed in terms of the Langmuir equation:

A=SKC_{eq}/(1+KC_{eq}) or C_{eq}/A=1/SK+C_{eq}/S (2)

where A is the amount of NPA adsorbed per unit volume of gel in the shrunken state, C_{eq} is the final equilibrium concentration in the solvent, S is the number of adsorbing sites per unit volume of gel or the amount of NPA necessary to saturate the adsorbing sites, and K is the affinity of one adsorption site by a NPA molecule. From the slope and the intercept at zero C_{eq} both S and K, and the overall affinity SK can be deduced.

Release Experiments. Gels loaded in the shrunken state with NPA were dried under vacuum for three days and then introduced in thermostatized vials containing 5 ml of 1 mM NaCl solution under stirring. The amount of NPA released at different times was determined by UV spectrophotometry (266 nm). The influence of temperature changes was analyzed between 20 to 60° C.

### EXAMPLE 4

Co-polymer Gel of Imprinter-Q with a Thermosensitive Monomer.

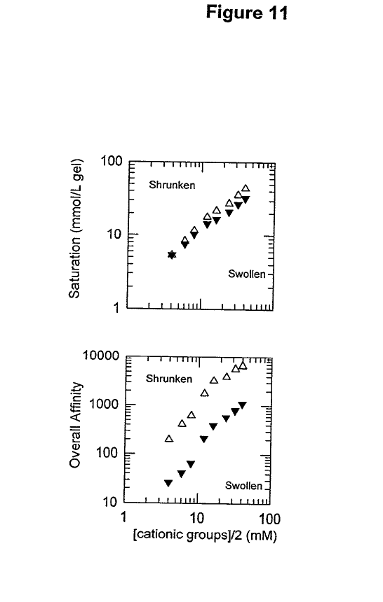

Co-polymer gels of Imprinter-Q (4 to 40 mM) and N-isopropylacrylamide (NIPA, 6M) weakly cross-linked with N,N′-methylenebis(acrylamide) (40 mM) were prepared by free radical polymerization in dimethylsulfoxide. The polymerization was carried out inside micropippetes (0.5 mm i.d.) at 60° C. for 23 h. After gelation, the gels were taken out the capillaries and washed. To break the 1,2-glycol bond, the gels were introduced into 0.1N NaIO₄ for 30 min, rinsed with deionized water, and transferred into 0.1N NaBH₄ aq. for 30 min. Finally, the gels were washed and dried under vacuum. Pieces of cylindrical gel of dry weight 10 to 20 mg were placed in 4 ml disodium nitrophthalate (8 μM to 0.5 mM). In the swollen (20° C.) and shrunken (60° C.) states, the saturation is approximately half the number of the cationic groups present in the gel (FIG. 11). This indicates that all the cationic groups participate in forming adsorption sites for disodium nitrophthalate. The affinity of the gels can be switched on and off by temperature changes. This shows the destruction and reformation of disodium nitrophthalate adsorption sites made of a pair of cationic groups.

### EXAMPLE 5

Adsorption of Disodium 5-Nitroisophthalate by Imprinter-Q Gels Compared to that by Gels Prepared with Randomly Distributed Cationic Groups.

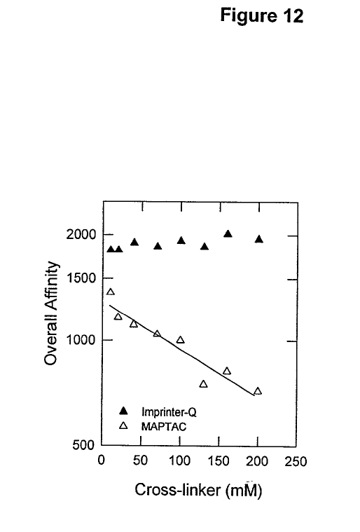

Following the procedure of Example 4, two types of gel containing Imprinter-Q/NIPA or methacrylamidopropyltrimethylammonium chloride (MAPTAC)/NIPA were synthesized. The concentration of cross-linker ranged between 10 to 200 mM. The adsorption of disodium nitrophthalate by both types of gel showed that, in the shrunken state, the affinity of the MAPTAC gels decayed exponentially as a function of cross-linker concentration (FIG. 12). The affinity of Imprinter-Q gels was much larger than that of the MAPTAc gels and did not decrease with the cross-linker concentration. This shows the difficulty of MAPTAC components to gather to adsorb the target, and that in contrast, the gels prepared with Imprinter-Q can memorize the position of the pairs of cationic groups after swelling and re-shrinking. Therefore, the excess of disodium nitrophthalate adsorption by Imprinter-Q gels comes from the successfully memorized pairs.

### EXAMPLE 6

Use of Imprinters in Chromatography

Imprinter-Q polymerizes with polymerizable components of methacrylate-base packings for HPLC chromatography. After breaking the 1,2-glycol bond, receptor sites formed by two cationic groups improve the ion-exchange ability of the column.

Incorporation by Reference

All of the patent and publication cited herein are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of preparing a gel polymer, comprising the steps of:
polymerizing monomers in the presence of an imprinter, wherein said imprinter comprises two ionic functional groups connected by a tether, and at least two polymerizable double bonds, to give a gel polymer; and
treating said gel polymer with a mixture comprising a reagent that breaks a covalent bond in the tether of said imprinter.

2. A gel polymer prepared according to the method of claim 1.

3. A monomer comprised of the following three parts: two or more polymerizable double bonds, two or more functional groups, and a breakable covalent bond that links said functional groups.

4. The monomer of claim 3, wherein the functional groups are selected from the group consisting of a quaternary ammonium group, a secondary amino group, a hydrophobic alkyl group, an aromatic group, an imidazole group, and a methylimidazolinium group.

5. A method of molecularly imprinting polymer networks without using a template, comprising the steps of co-polymerizing the monomer of claim 3 and subsequently breaking the breakable covalent bond.

6. The monomer of claim 3, wherein the breakable bond is a 1,2-glycol bond.

7. The monomer of claim 5, wherein the monomer is 2,3-Dihydroxy-N,N,N′N′-tetramethyl-N,N′-bis {3-[(2-methylacryloyl)amino]propyl}-1,4-butanediaminium dihalide.

8. A method of molecularly imprinting polymer networks without using a template, comprising the steps of co-polymerizing the monomer described in claim 5 and subsequently breaking the breakable covalent bond.

9. A polymer network prepared by the method of claim 7.

10. A method of molecularly imprinting polymer networks without using a template, comprising the steps of co-polymerizing the monomer described in claim 6 and subsequently breaking the breakable covalent bond.

11. A polymer network prepared by the method of claim 9.

12. Separation materials comprising the gel polymer of claim 2.

13. Separation materials comprising the polymer network of claim 8.

14. Separation materials comprising the polymer network of claim 10.

15. Sensors comprising the gel polymer of claim 2.

16. Sensors comprising the polymer network of claim 8.

17. Sensors comprising the polymer network of claim 10.
